(19) United States
(12) Patent Application Publication
(10) Pub. No.: US 2008/0146908 A1
(43) Pub. Date: Jun. 19, 2008

(54) Lithotripter with stone tracking and locking localization system

(76) Inventor: Jung-Chu Wu, Taipei City (TW)

Correspondence Address: JUNG-CHU WU, 235 Chung-Ho, Box 8-24, Taipei, omitted

(21) Appl. No.: 11/584,930

(22) Filed: Oct. 24, 2006

(51) Int. Cl. A61B 5/05

(52) U.S. Cl. 600/407

(57) ABSTRACT

A lithotripter with stone tracking and locking localization system comprises an X-ray machine, an ultrasound scanner, an ultrasound probe, a movable platform, a monitor, a system controller and a stone tracking and locking localization system. The X-ray generator is mounted on the end of rotational arm, capable of illuminating across a range from 0 to 30 degrees, especially at 0 and 30 degrees, whereby the position of stones embedded in a patient's body can be located in a three-dimensional way. The ultrasound probe is located under the movable platform and shock-cup, whereby the image of a stone embedded in the patient can be displayed on the monitor after the ultrasound probe moves and contacts the surface of body. The stone tracking and locking localization system then lock on the position of the stone. And starts the tracking process by driving the movable platform to always keep the stone in the focal point F2 or using the locking process to pulverize the stone which is on the focal point F2 (if the stone moves out of the focal point F2, the shock waves will not be triggered until the stone moves into the F2 again).

### FIELD OF THE INVENTION

The present invention relates to lithotripters, and more particularly to a lithotripter with stone tracking and locking localization system comprising an X-ray machine, an ultrasound scanner, an ultrasound probe, a movable platform, a monitor, a system controller and a stone tracking and locking localization system. The X-ray generator is capable of exposing a stone across a range from 0 to 30 degrees(especially at 0 and 30 degrees), whereby the position of a stone embedded in a patient's body can be located in a three-dimensional way. The stone tracking and locking localization system can always lock on the position of the stone, and a system controller drives the platform so as to align the stone with the focal point (F2) for pulverizing the stone by focusing shock waves.

### BACKGROUND OF THE INVENTION

Because the styles of our daily diet today, the health problem of kidney stone has become more and more common, resulting in even life threatening hazard to a person and huge burdens to the society.

In the past twenty years, the most common medical treatment of stones in human bodies has changed from destructive operation to using an extracorporeal lithotripter to pulverize the stones.

Not only in tackling kidney stones, lithotripters are also used to pulverize the stones formed within the bladder and the urethra.

A lithotripter uses shock waves focusing process where the shock waves pass through the medium of water and human tissues and converge on a stone in the human body. The pressure wave convergence results in the highest pressures being found in the vicinity of stone (focal point F2). The shock waves may hurt the tissues near the stone. Therefore, the tracking, the locking, the focusing efficiency and the effect of treatment are directly related.

However, the safety and effect of treatment are affected by the movement of the stone following the vibrations of the internal organs due to respiration or other causes. As a consequence, the stone is easy to leave where the waves are already focused, and harmless and healthy tissues may be hit and damaged.

Therefore, the efficiency of tracking and locking localization system is important.

### SUMMARY OF THE INVENTION

The primary objective of the present invention is to provide a lithotripter with stone tracking and locking localization system that integrates an X-ray machine, an ultrasound scanner, an ultrasound probe, a movable platform, a monitor, a system controller and a stone tracking and locking localization system. The X-ray machine is capable of illuminating the stones embedded in a patient's body in a three-dimensional way, whereby the positions of the stones will be displayed on the monitor and be located by the system controller, and the ultrasound probe located under the movable platform can move lateral and up down to contact the surface of body, whereby the image of a stone embedded in the patient can also be displayed on the monitor.

The secondary objective of the present invention is to provide a lithotripter with stone tracking and locking localization system capable of carrying out a tracking and locking process comprising the steps of initial setup, image capturing, stone detection, image comparison and positioning the stone. Thereby, the stone tracking and locking localization system then lock on the position of the stone, and the controller drives the platform so as to align the stone with the focal point F2 for pulverizing the stone by focusing shock waves.

It is a further objective of the present invention that the tracking and locking process of the present invention further comprises the step of using the stone shadow to assist the localization, whereby the precision of a stone position will be enhanced.

It is another objective of the present invention that the center lines of probe, shock cup and X-ray generator of the present invention aiming at the focal point (F2).

To achieve the above objectives, a lithotripter with stone tracking and locking localization system comprises an X-ray machine, an ultrasound scanner, an ultrasound probe, a movable platform, a monitor, a system controller and a stone tracking and locking localization system. The X-ray generator located under the movable platform is capable of illuminating across a range from 0 to 30 degrees, whereby the position of stones embedded in a patient's body can be located in a three-dimensional way. The ultrasound probe is located under the movable platform, whereby the image of a stone embedded in the patient can be displayed on the monitor. The stone tracking and locking localization system then lock on the position of the stone, and system controller drives the stone to the focal point F2 for the shaker for pulverizing the stone by focusing shock.

### BRIEF DESCRIPTION OF THE DRAWINGS

FIG. 1(A) is a perspective view of a lithotripter with stone tracking and locking localization system of the present invention.

FIG. 1(B) is a side exploded view of the electromagnetic shock wave generator of a lithotripter of the present invention.

FIG. 2 is another perspective view of the lithotripter in FIG. 1.

FIG. 3 is a side view of the lithotripter in FIG. 1.

FIG. 4 illustrates the rotation of the C-arm of the X-ray machine 1 of the lithotripter in FIG. 1.

FIG. 5 is the flow chart of the stone tracking and locking process of the lithotripter with stone tracking and locking localization system.

FIG. 5-1 is the flow chart of the steps of stone detection.

FIG. 5(A) is the local peek detection.

FIG. 5(B) is the flow chart of the step of frame matching.

FIG. 5(B-1) is a photo of the ultrasonic image produced during the step of frame matching.

FIG. 5(C) is the flow chart of the step of assisting localization by stone shadows.

FIG. 5(D) is the flow chart of the steps of stone detection and assisting detection by stone shadows.

FIG. 6 is the system diagram of the lithotripter in FIG. 1.

FIG. 7 is an ultrasonic image produced from the ultrasonic scanning of the present invention.

FIG. 8 shows the defined region of interest (ROI) of an ultrasonic image.

FIG. 9 shows the defined region of interest (ROI) of another ultrasonic image.

The various objects and advantages of the present invention will be more readily understood from the following detailed description when read in conjunction with the appended drawings.

### DETAILED DESCRIPTION OF THE PREFERRED EMBODIMENTS

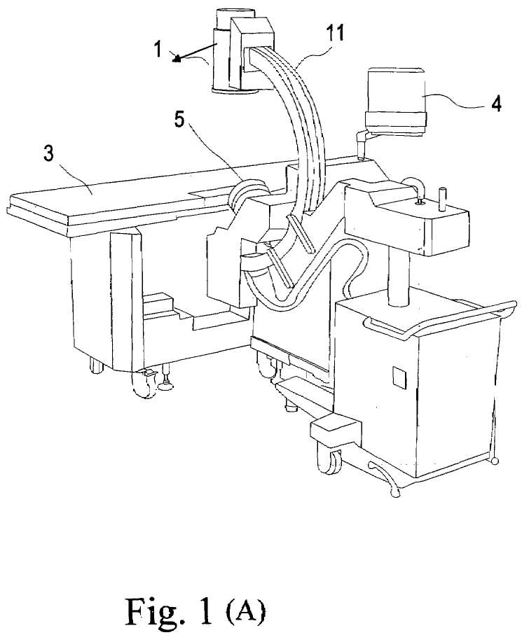

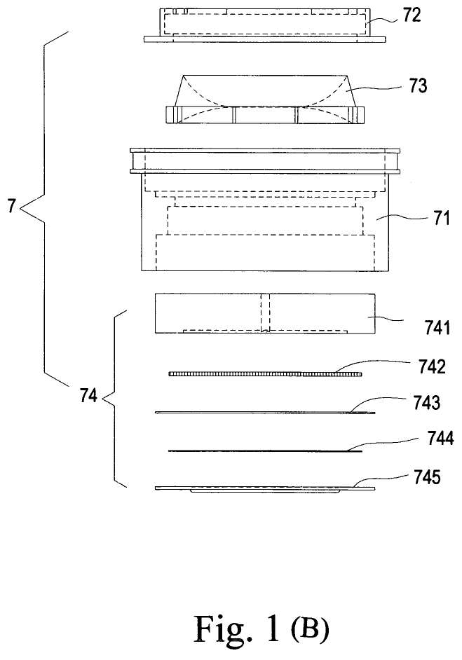

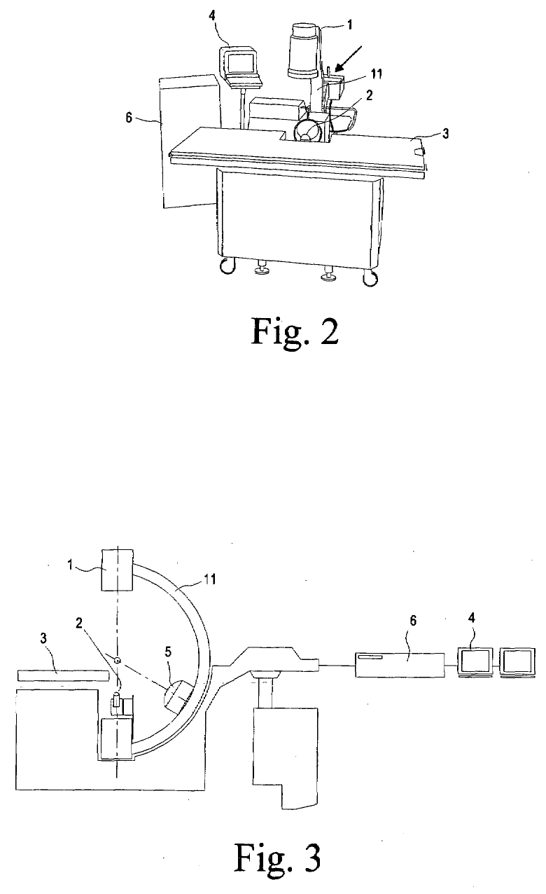

Referring to FIG. 1(A) and FIG. 2, a lithotripter with stone tracking and locking localization system comprises X-ray machine 1, an ultrasound scanner 4, a movable platform 3, a monitor 8, a shock cup 5 and a system controller 6. The X-ray generator 12 is mounted on the end of a rotational arm 11. The shock-cup 5 is located at a predetermined position at the upper side of the X-ray generator 12. The ultrasound probe 2 is located above the X-ray generator 12, whereby the ultrasound probe 2 can be driven by the system controller 6 to move for displaying the stone image.

Referring to FIG. 1(B), the shock-cup further includes an electromagnetic shock wave generator 7 consisting of a shock-cup mount 71, a shock wave disk assembly mount 72 located on the bottom to fix the shock wave disk assembly 74 on the shock-cup mount 71, a focusing double-concave lens 73 and a shock wave disk assembly 74 located under the shaker 5. The shock wave generating unit 74 further comprises an insulating ceramic 741, a high-voltage coil 742, a insulating membrane 743, a metallic membrane 744 and a rubber membrane 745, whereby an electric current will go through the high-voltage coil 742 for producing a magnetic field that in turn drives the metallic film 744 to beat the water within the shock-cup 5, whereby shock waves will be generated and propagate outwardly to focus on the focal point F2 to pulverize a stone in the body.

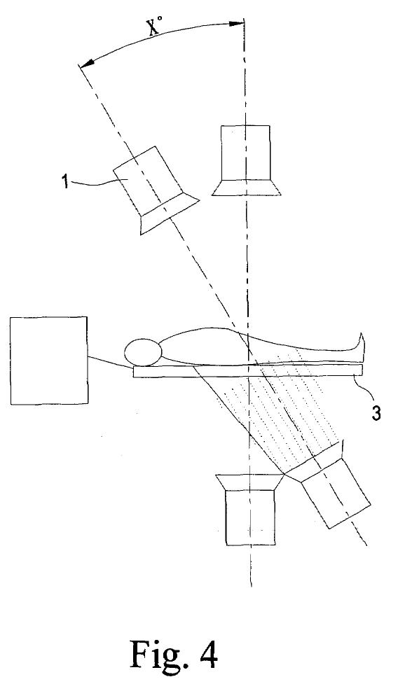

Referring to FIGS. 3 and 4, the lithotripter with stone tracking and locking localization system has the X-ray generator 12 mounted on the bottom end of a rotational arm 11, which can rotate angles from 0 to 30 degrees especially at 0 and 30 degrees by the system controller 6. Thereby, the stone position in the patient can be determined. The projections axes of the X-ray generator 12, the ultrasound probe 2 and the shock-cup 5 are intersected in their center lines to define the target F2 of pulverize the stone. After the X-ray generator 9 locates the stone area, the ultrasound probe 2 will shift to grab an image of the stone position on the monitor 8.

Further, system controller 6 is connected drivers, AC motors, encoders and reduction gears, whereby the movable platform will move in three dimensions and the stone position of a patient thereon will be transported to the focal point F2 of the shock waves.

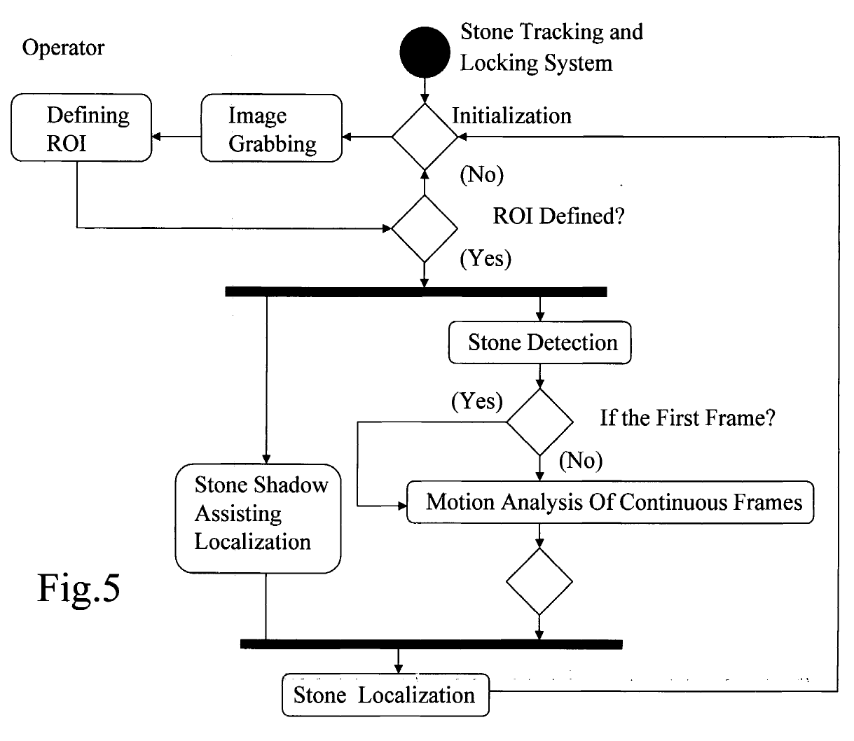

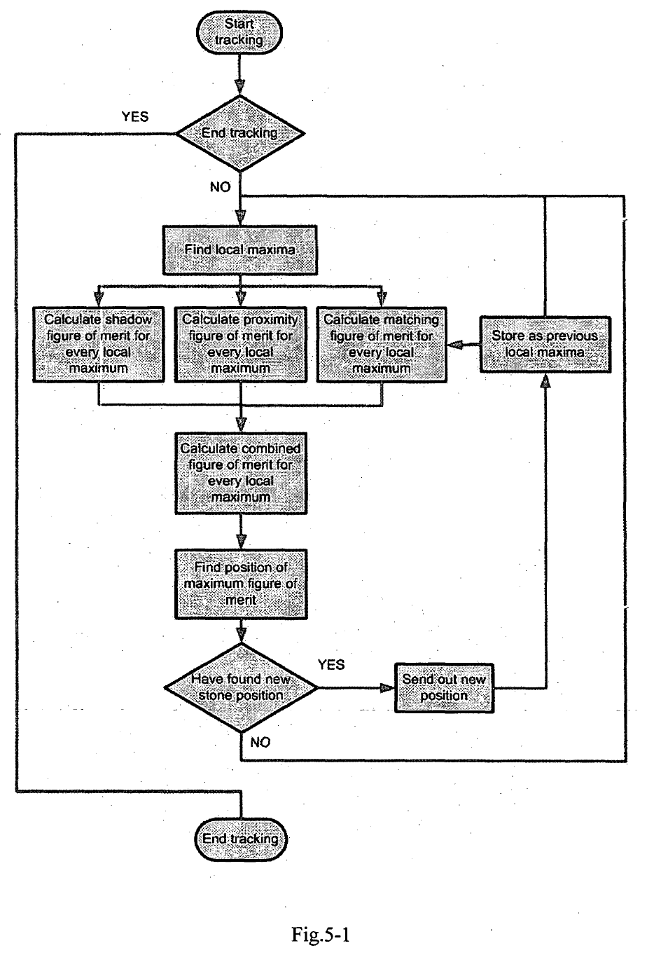

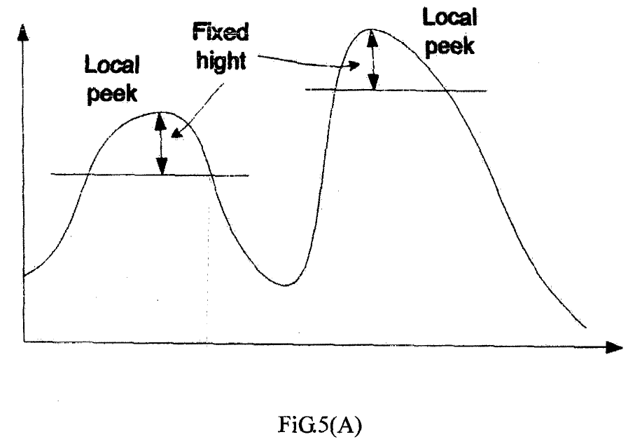

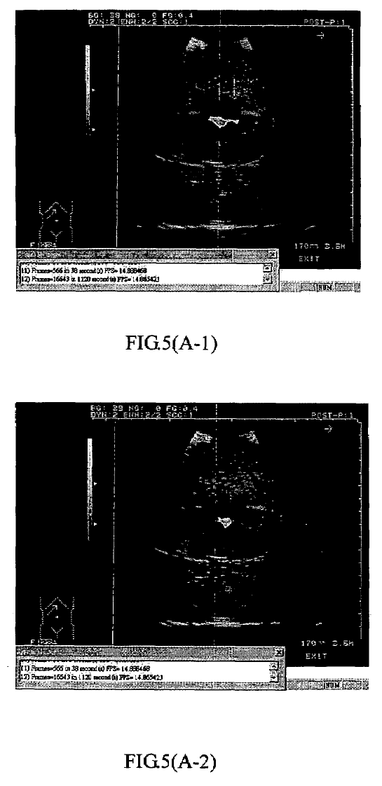

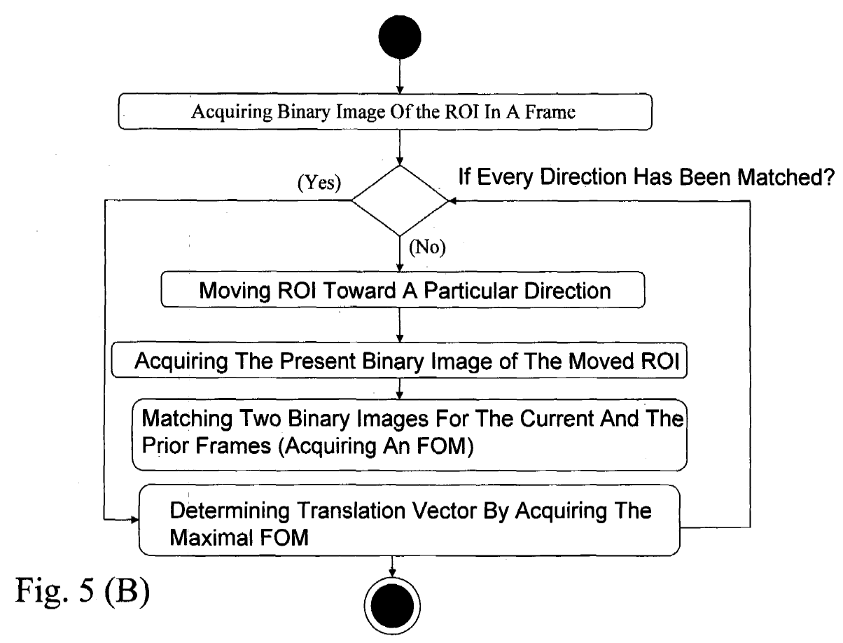

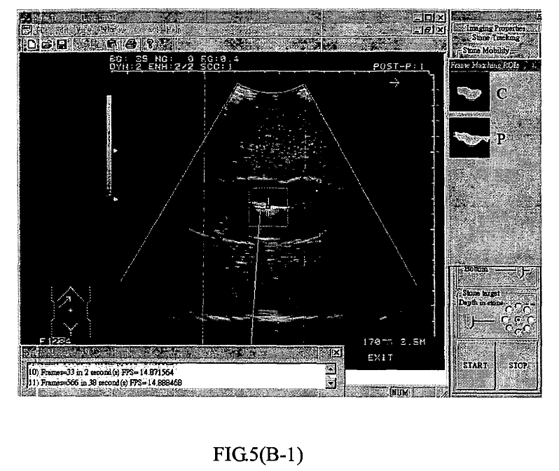

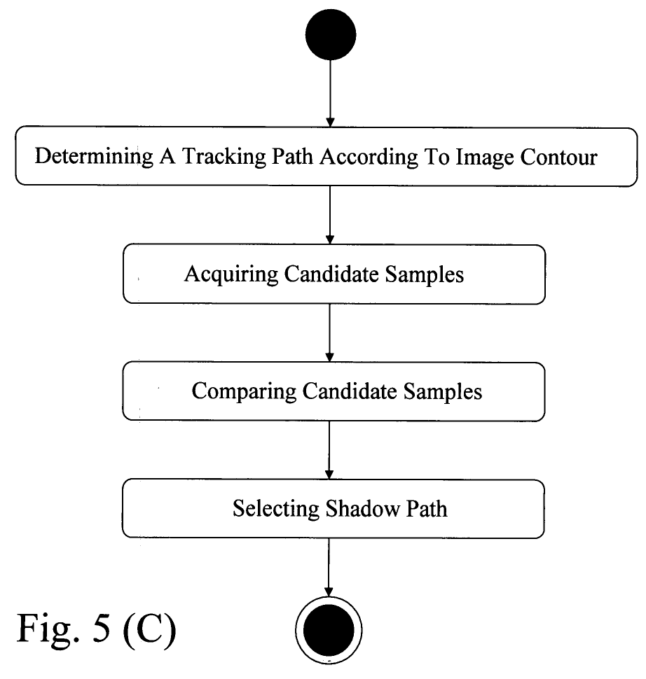

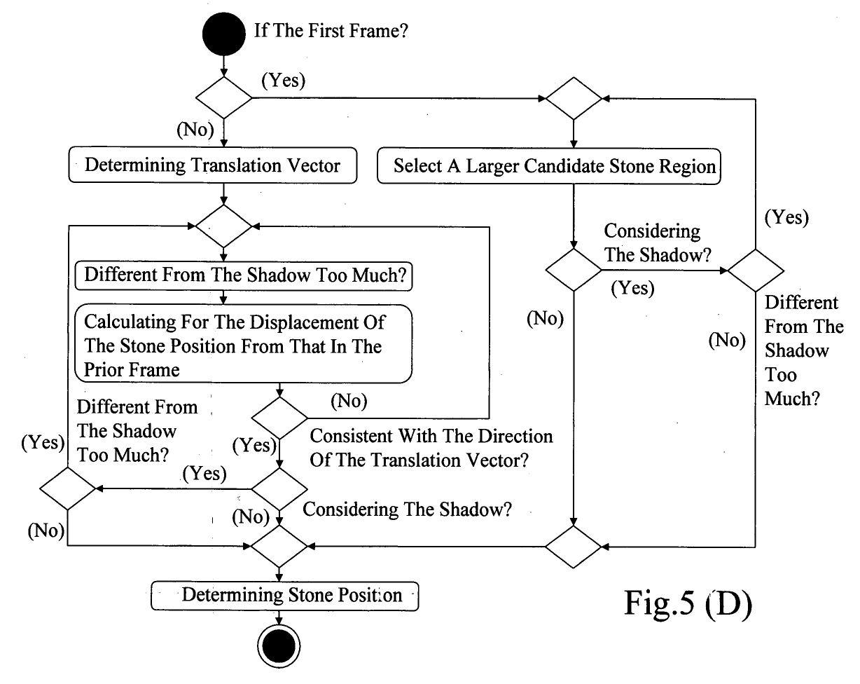

Referring to FIG. 5, the tracking/locking procedure of the present invention comprises the steps of:

(A) Initial setup: Assign the memory for tracking and locking image buffer in advance. And set up the initial values of various operation parameter. The image capture card is initialized and ready for receiving commands.

(B) Image grabbing: Images are taken by the technique of multithread and then stored in the buffer. The commands entered at the graphical user interface are processed by an event-driven scheme, whereby the response time to a user's command will be reduced. Further, the technique of double buffering is used to avoid missing the images and assure immediate tracking and locking.

(C) Stone detection: Using a brightness peek detection (local maxima peek). The peeks areas are extended to a predefined brightness level to form larger areas. Small isolated areas of just few pixel are excluded. The resulted regions are then considered as possible stone locations as shown in FIG. 5(A-1) and 5(A-2).

(D) frame matching: Binary images produced according to the binary image processing of the prior frame and the subsequent one are matched, whereby the white overlapped regions will be weighted and the non-overlapped regions will be given less weight. The addition of all weight values is called matching value. The ROI is then moved in various directions to get various matching values; the direction of highest matching value will be used to determine the translation vector of the ROI.

(E) Stone location: The derived translation vector is added to the stone position previously determined. The new location is the center of the marked candidate location of the stone that is more close to the calculated new position.

Referring to FIG. 5-1, the stone detection procedure of the present invention further comprises the steps of:

(A) Find local maxima;

(B) Calculate shadow, proximity and matching figure of merit for every local maximum;

(C) Calculate combined figure of merit for every local maximum;

(D) Find position of maximum figure of merit;

(E) Have found new stone position;

(F) Send out new position;

(G) Store as previous local maxima;

(H) Repeat the (A) to (G) for the next image.

In the step of a user's intervening, the user only uses mouse to mark the ROI. After the ROI is defined, all the gray-level values of the pixels in the ROI are calculated to obtain local maxima regions.

A simple morphological processing is then used to remove very small regions. In the binary diagram the white areas are possible stone regions, whose contours are much simplified, as shown in FIG. 5(A-1) and FIG. 5(A-2). The largest whiter area in the ROI that does not coincide with the border of the ROI is interpreted to be the stone region.

In most of the cases, the stones in the ROI can be correctly located. However, the stone will move with the movements of the internal organs, resulting in vibrations of the stone region. Therefore, it is important to track and lock the movement of the stone region using the method of frame matching.

Referring to FIG. 5(B), the above mentioned procedure of frame matching comprises the steps of:

(A) retrieving the binary image of the ROI in the prior frame;

(B) moving the ROI in any direction;

(C) deriving the binary image of the moved ROI;

(D) comparing the binary images of the moved ROI and the ROI in the prior frame;

(E) moving the ROI in other directions and obtaining respective matching values to determine the FOM (figure of merit) the moving of direction of the stone.

The detailed process is described as follows. The FOM is used as the reference image frame, and when the pixels of stone region in the prior frame matches the ones of current stone region, the matching max value of the FOM is calculated by Equation (1):

$$FOM = {\sum }_{for x , y and i , j where P and C overlap} W \cdot [ {P}_{x , y} ( AND ) {C}_{i , j} ] \qquad ( 1 )$$

where P is the prior frame, C is the current frame and W is the weight value.

When the calculation is being performed, the ROI in the current frame is shifted in various directions to acquire a set of binary images. The set of binary images are compared against the prior image to get FOM values in various directions. The one having the highest FOM value is used to determine the moving direction.

The derived translation vector is added to the stone position previously determined, so that the new stone position after it moved can be determined.

Referring to FIG. 5(B-1), two ultrasonic images produced by the tracking/locking system of the present invention, one is a prior image (P) and the other is the current image (C), are the binary images of the ROI. The large region in the prior image (P) is the stone location, whereas the smaller region in the current image (C) is also the stone location but it shrinks due to the surrounding tissue movement. The procedure of frame matching will determine the translation vector of the stone and therefore can precisely determine the stone location.

Therefore, the procedure of frame matching will precisely determine the moving direction of the stone and its location, whereby the problem of stone movement due to internal organ movements will be confined, and whereby the accuracy of pulverizing the stone will be improved.

To enhance the precision of stone position recognition, the present invention develops a system of assisting localization via stone shadow. During the imaging process, the ultrasound waves can't pass through dense material such as stones and bones. So a long shadow appears after a renal stone. The pixels in the shadow have a lower gray level values than those of the surrounding tissues. Also a stone has higher gray level values due to the strong reflection of ultrasound waves from the stone. However, if a region has a high value of gray level and there is a long shadow after it, the area is usually regarded as a stone.

Referring to FIG. 5(C), the procedure of assisting stone tracking and locking by shadows of the present invention comprises the steps of:

(A) determining shadow paths based on the contour in an image;

(B) retrieving candidate samples;

(C) comparing candidate samples;

(D) selecting a shadow path;

(E) determining stone location.

To carry on the procedure, a shadow path is pre-defined. Shadow starting positions only exist within ROI. If the stone shadow coincides with the tracking path, the matching result is optimal. Since a shadow sample has two characteristics: the stone portion and the shadow portion.

Referring to FIG. 5(D) for a system block diagram of the procedures of the stone tracking/locking localization and assisting positioning by stone shadows, wherein the procedure of assisting positioning by stone shadows can go with the procedure of frame matching at the same time for a more precise positioning of the stone. After a user defines the region of interest (ROI) of a stone, the procedure of stone tracking/locking determines the stone position, and the procedure of assisting positioning by stone shadows, coupled with that of frame matching determine the position of a tone in motion with a desired precision.

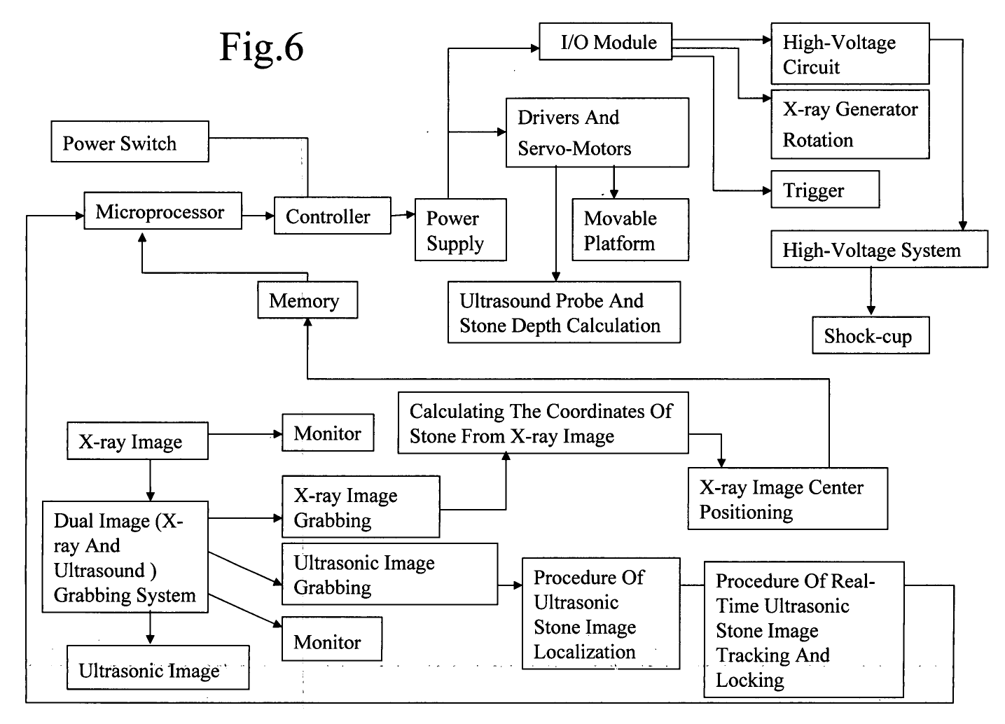

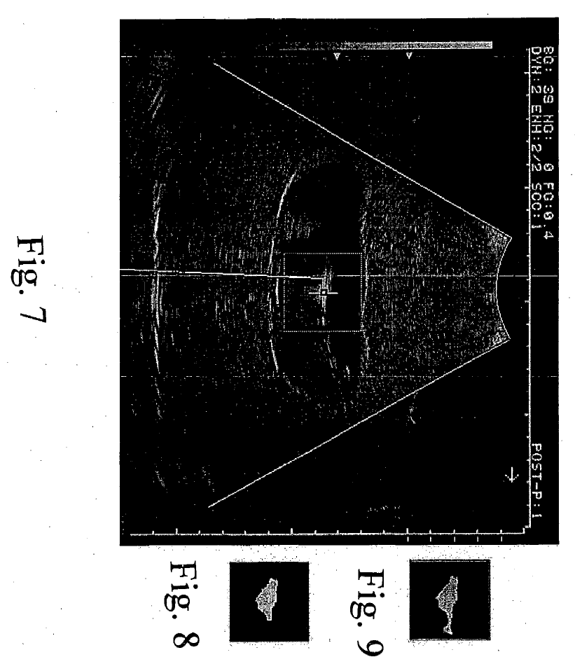

Referring to FIG. 6 for a system block diagram of the lithotripter with stone tracking and locking system, wherein an operation starts at power activation whereby the controller will turn on the power supply. Accordingly, an I/O module drives the X-ray generator to rotate an angular range from 0 to 30 degrees (especially at 0 and 30 degrees)for locating the stone area in a patient's body. And the ultrasound probe, driven by a driving control device and servo motors, scans the patient for determining the depth of the stone. The procedures of ultrasonic stone localization and ultrasonic stone tracking and locking are then performed to mark the stone position, as shown in FIGS. 7, 8 and 9. The series of frames marked with the stone position are matched to determine the translation vector of the stone. And starts the tracking process by driving the movable platform to always keep the stone in the focal point F2 or using the locking process to pulverize the stone which is on the focal point F2 (if the stone is moving out of the focal point F2, the shock waves will not be triggered until the stone moves into the F2 again).

The present invention is thus described, and it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A lithotripter with stone tracking and locking localization system, comprising:
An X-ray machine;
an X-ray generator mounted on said X-ray machine being capable of illuminating across an angular range from 0 to 30 degrees, especially at 0 and 30 degrees, whereby the three-dimensional position of a stone embedded in a patient's body will be determined;
a movable platform for placing said patient;
an ultrasound scanner; and
an ultrasound probe located under said movable platform, can move lateral and up-down to contact the surface of body, whereby the image of a stone embedded in the patient can also be displayed on the monitor;
whereby a stone tracking and locking localization system will lock on said stone, and whereby a system controller will drive said movable platform so as to align said stone with said shock-cup for pulverizing said stone by focusing shock waves.

2. The lithotripter with stone tracking and locking localization system of claim 1 wherein said shock-cup includes an shock wave generator module; said shock wave generator module further comprises a disk assembly mount, a double concave lens a shock-cup mount and a shock wave disk assembly, whereby an electric current will go through a high-voltage coil for producing magnetic field therein that in turn drives a metallic membrane to beat the water in said shock-cup, and whereby shock waves produced will be focus on the focal point F2 by a said concave lens and sent out to pulverizing said stone in a human body.

3. The lithotripter with stone tracking and locking localization system of claim 2 wherein said shock wave disk assembly comprises an insulating mount, said high-voltage coil, a insulating membrane, said metallic membrane and a rubber thin film.

4. The tracking and locking method of a lithotripter with stone tracking and positioning system, comprising the steps of:
(A) initialization: setting initial values of various operation parameters; initializing an image capture card to wait for a command;
(B) image grabbing: capturing images by a multi-thread technique and saving said images in a buffer;
(C) stone detection: Using a brightness peek detection (local maxima peek). The peeks areas are extended to a predefined brightness level to form larger areas. Small isolated areas of just few pixel are excluded. The resulted regions are then considered as possible stone locations
(D) frame matching: comparing binary images corresponding to a current image and a prior image; weighting overlapped regions and non-overlapped regions of two binary images differently; acquiring a translation vector from said prior image to said current image; and
(E) stone location: adding said translation vector to a prior stone position to get a new stone position.

6. The tracking and locking method of a lithotripter with stone tracking and locking localization system of claim 4 wherein the step of frame matching utilizes a formula to derive a matching value, called FOM, fro determining if two frame are matched; said formula being:
$$FOM = {\sum }_{for x , y and i , j where P and C overlap} W \cdot [ {P}_{x , y} ( AND ) {C}_{i , j} ]$$
where W is a weight value, P is a prior frame and C is a current frame.

7. The tracking and locking method of a lithotripter with stone tracking and locking localization system of claim 6 wherein the step of frame matching utilizes a scheme of determining a stone moving direction by finding the maximal matching value (FOM) of a pair of a prior binary image and a current binary image, produced by moving a ROI in each of said prior and current frames in a possible direction; said ROI being moved in various directions;

8. The tracking and locking method of a lithotripter with stone tracking and locking localization system, comprising the steps of:
(A) initialization: setting initial values of various operation parameters; initializing an image capture card to wait for a command;
(B) image grabbing: capturing images by a multi-thread technique and saving said images in a buffer;
(C) stone detection: Using a brightness peek detection (local maxima peek). The peeks areas are extended to a predefined brightness level to form larger areas. Small isolated areas of just few pixel are excluded. The resulted regions are then considered as possible stone locations.
(D) frame matching: comparing binary images corresponding to a current image and a prior image; weighting overlapped regions and non-overlapped regions of two binary images differently; acquiring a translation vector from said prior image to said current image; and
(E) stone location: adding said translation vector to a prior stone position to get a new stone position;
(F) stone shadow assisting localization: after said step of stone localization, synchronically determining a shadow path based on the contour in an image; retrieving and comparing candidate samples; matching said candidate samples; selecting a shadow; assuring stone position.

9. the tracking and locking localization of claim 8 wherein said step of stone detection comprising the steps of:
(A) Find local maxima;
(B) Calculate shadow, proximity and matching figure or merit for every local maximum;
(C) Calculate combined figure of merit for every local maximum;
(D) Find position of maximum figure of merit;
(E) Have found new stone position;
(F) Send out new position;
(G) Store as previous local maxima;

10. The tracking method of a lithotripter with stone tracking and locking localization system of claim 8 wherein said step of stone shadow assisting localization uses a scheme of defining tracking paths first and matching said shadow path and said stone shadow; said a stone shadow being simulated by finding the most probable section along said shadow path first and then the most probable point in said section

11. The tracking method of a lithotripter with stone tracking and locking localization system of claim 10 wherein said shadow path starts at an appropriate distance from an ultrasound probe; a set of candidate samples being collected behind said shadow path; an average gray-level value of each of said candidate samples being calculated and reduced from a corresponding peak value; a stone being identified by a maximal difference between said gray-level peak value and said gray-level average value.

12. The tracking method of a lithotripter with stone tracking and locking localization system of claim 8 wherein said stone location is driving the movable platform to always keep the stone in the focal point F2 or using the locking process to pulverize the stone which is on the focal point F2, if the stone moves out of the focal point F2, the shock waves will not be triggered until the stone moves into the F2 again.
